(19) United States
(12) Patent Application Publication
(10) Pub. No.: US 2025/0325378 A1
(43) Pub. Date: Oct. 23, 2025

(54) EXPANDABLE IMPLANT WITH PIVOTING CONTROL ASSEMBLY

(71) Applicant: Life Spine, Inc., Huntley, IL (US)

(72) Inventors: Garrett Lauf, Hampshire, IL (US); Brett Nowak, Lake in the Hills, IL (US); Daniel Predick, Wheat Ridge, CO (US); Paul Christopher Zakelj, Chicago, IL (US)

(21) Appl. No.: 19/256,679

(22) Filed: Jul. 1, 2025

Related U.S. Application Data
(63) Continuation-in-part of application No. 17/990,101, filed on Nov. 18, 2022, now Pat. No. 12,364,610.
Application No. 17/990,101 is a continuation of application No. 17/014,546, filed on Sep. 8, 2020, now Pat. No. 11,554,020.

(51) Int. Cl. A61F 2/44; A61F 2/30

(52) U.S. Cl. CPC A61F 2/442; A61F 2/30744; A61F 2002/30224; A61F 2002/30405; A61F 2002/30528; A61F 2002/30538

(57) ABSTRACT

An expandable implant comprising a first support comprising a control aperture defined by and extending through the first support, a second support movably coupled to the first support, and a control assembly. The control assembly comprising a control shaft, a base member received on the control shaft, the base member configured to translate along the control shaft, and a pivot member pivotally coupled to the base member and movably coupled with the control aperture, where translation of the base member along the control shaft causes the pivot member to pivot relative to the base member, and the first support to move relative to the second support to change an angle between the first support and the second support.

### BACKGROUND

This application is a continuation-in-part of U.S. patent application Ser. No. 17/990,101, filed Nov. 18, 2022, which is a continuation of U.S. patent application Ser. No. 17/014,546, filed Sep. 8, 2020, which are incorporated herein by reference in their entireties.

### BACKGROUND

The present disclosure relates generally to expandable implants usable in connection with the spine or other parts of the human anatomy. Certain implants are expandable, in that the implants may, for example, have a variable height dependent upon a degree of expansion.

### SUMMARY

At least one embodiment relates to an expandable implant. The expandable implant includes a lower support; an upper support pivotally coupled to the lower support and including a control channel; and a control assembly. The control assembly includes a control shaft coupled to the lower support; and a control member coupled to the control shaft and configured to move along the control shaft. The control member includes a base member and a pivot member pivotally coupled to the base member, the pivot member configured to move within the control channel. Movement of the control member along the control shaft causes the pivot member to pivot relative to the base member, and the upper support to pivot relative to the lower support.

Another embodiment relates to an expandable implant. The expandable implant includes a first support; an second support pivotally coupled to the first support; a control shaft rotatably coupled to the first support; and a control member coupled to the control shaft and configured to move along the control shaft such that movement of the control member along the control shaft cause pivotal movement of the second support relative to the first support, a portion of the control member configured to rotate relative to the second support as the control member moves along the control shaft.

Another embodiment relates to an expandable implant. The implant includes a lower support having a first lower surface, a first upper surface, an access bore configured to receive an expansion tool, and an inner housing that defines a central aperture extending between the first lower surface and the first upper surface, an upper support having a second upper surface, a second lower surface, a control channel, and a rear aperture extending between the second upper surface and the second lower surface, wherein the upper support is pivotally coupled to the lower support, the implant is configured to expand between a first, collapsed position and a second, expanded position such that pivotal movement of the upper support relative to the lower support changes an angle defined between the first lower surface and the second upper surface as the implant expands, and at least a portion of the inner housing is received by the rear aperture the first, collapsed position, a control shaft rotatably coupled to the lower support, wherein the control shaft includes a head configured to receive the expansion tool, wherein manipulation of the expansion tool causes the implant to expand, and wherein the central aperture is located between the head and the access bore, and a control member threadingly coupled to the control shaft, the control member includes a base member threadingly coupled to the control shaft and rotatably fixed relative to the lower support, a first pivot member pivotally coupled to a first side of the base member and slidingly received in the control channel, a second pivot member pivotally coupled to a second side of the base member opposite the first side and slidingly received in the control channel.

Another embodiment relates to an expandable implant. The expandable implant comprises a first support comprising an exterior surface configured to engage bone and a control aperture, the control aperture defined by the first support and extending through the exterior surface. The implant further comprises a second support movably coupled to the first support and comprising a recess defined by a peripheral wall, and a control assembly. The control assembly comprises a control shaft; and a base member received on the control shaft, where the base member is configured to translate along the control shaft within the recess, and where translational movement of the base member along the control shaft and within the recess is limited by a first portion of the peripheral wall. The control assembly further includes a pivot member pivotally coupled to the base member and configured to move within the control aperture, where translation of the base member along the control shaft causes the pivot member to pivot relative to the base member, and the first support to move relative to the second support to change an angle between the first support and the second support.

Another embodiment relates to an expandable implant. The expandable implant comprising a first support defining a control aperture extending through the first support, and a second support movably coupled to the first support, where the second support comprises a first end having an access bore and a second end opposite the first end and comprising a recess. The implant further comprise a control assembly comprising a control shaft movably coupled with the second support, where the access bore provides tool access to the control shaft via the access bore, a base member received on the control shaft, and a pivot member pivotally coupled to the base member and movably coupled with the control aperture. The implant further comprises a cap received by the recess that is configured to receive an end portion of the control shaft to position the control shaft relative to the second support, and where translation of the base member along the control shaft causes the pivot member to pivot relative to the base member, and the first support to move relative to the second support to change an angle between the first support and the second support.

Another embodiment relates to an expandable implant. The expandable implant comprises a first support defining a control aperture extending through the first support, and a second support movably coupled to the first support, where the second support comprises a recess defined by a boundary wall. The implant also comprises a control assembly comprising a control shaft movably coupled with the second support, a base member movably received on the control shaft, where movement of the base member along the control shaft is limited by a first portion of the boundary wall, and a pivot member pivotally coupled to the base member and movably coupled with the control aperture. The implant further comprises a cap received by a lower recess of the second support, where the cap configured to receive the control shaft to position the control shaft relative to the second support, and where translation of the base member along the control shaft causes the pivot member to pivot relative to the base member, and the first support to move relative to the second support.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE FIGURES

FIG. 1 is a perspective view of an implant in a collapsed position, according to one embodiment.

FIG. 2 is a perspective view of the implant of FIG. 1 in an expanded position, according to one embodiment.

FIG. 3 is a side view of the implant of FIG. 1 in the collapsed position, according to one embodiment.

FIG. 4 is a cross-section view of the implant of FIG. 1 in a collapsed position, according to one embodiment.

FIG. 5 is a side view of the implant of FIG. 1 in an expanded position, according to one embodiment.

FIG. 6 is a side view of the implant of FIG. 1 in an expanded position, according to one embodiment.

FIG. 7 is an exploded view of the implant of FIG. 1, according to one embodiment.

FIG. 8 is another exploded view of the implant of FIG. 1, according to one embodiment.

FIG. 9 is a side cross-section view of an upper support of the implant of FIG. 1, according to one embodiment.

FIG. 10 is a top view of the implant of FIG. 1, according to one embodiment.

FIG. 11 is a bottom view of the implant of FIG. 1, according to one embodiment.

FIG. 12 is a front view of the implant of FIG. 1, according to one embodiment.

FIG. 13 is a rear view of the implant of FIG. 1, according to one embodiment.

FIG. 14 is a perspective view of an implant in a collapsed position, according to one embodiment.

FIG. 15 is a perspective view of the implant of FIG. 14 in an expanded position, according to one embodiment.

FIG. 16 is a side view of the implant of FIG. 14 in a collapsed position, according to one embodiment.

FIG. 17 is a cross-section view of the implant of FIG. 14 in a collapsed position, according to one embodiment.

FIG. 18 is a side view of the implant of FIG. 14 in an expanded position, according to one embodiment.

FIG. 19 is a cross-section view of the implant of FIG. 14 in an expanded position, according to one embodiment.

FIG. 20 is an exploded view of the implant of FIG. 14, according to one embodiment.

FIG. 21 is another exploded view of the implant of FIG. 14, according to one embodiment.

FIG. 22 is a side cross-section view of an upper support of the implant of FIG. 14, according to one embodiment.

FIG. 23 is a top view of the implant of FIG. 14, according to one embodiment.

FIG. 24 is a bottom view of the implant of FIG. 14, according to one embodiment.

FIG. 25 is a front view of the implant of FIG. 14, according to one embodiment.

FIG. 26 is a rear view of the implant of FIG. 14, according to one embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring generally to the figures, various embodiments of an expandable implant are disclosed herein. The expandable implant may be usable in connection with the spine (e.g., between vertebral bodies) or other parts of the human body. In some embodiments, the implant provides a lumbar interbody expandable implant that expands in a lordotic fashion. The implant may include an upper support hingedly or pivotally coupled to a lower support, such that an amount of lordosis provided by the implant can be adjusted as desired. A control assembly may include a control shaft and a control member mounted to the control shaft. One or more pivoting members are pivotally coupled to the control member and move within one or more control channels in the upper implant. In one embodiment, rotation of the control shaft causes translation of the control member along the control shaft relative to the lower support. As the control member translates, ramp surfaces on the pivoting member(s) slidingly engage corresponding ramp surface(s) on the upper support to cause expansion or contraction of the implant (e.g., to move the implant between a collapsed position and an expanded position, and intermediate positions therebetween).

The implants disclosed herein may be made of any suitable materials, including a variety of metals, plastics, composites, or other suitable bio-compatible materials. In some embodiments, some or all of the components of the implants disclosed herein may be made of the same material, while in other embodiments, different materials may be used for different components.

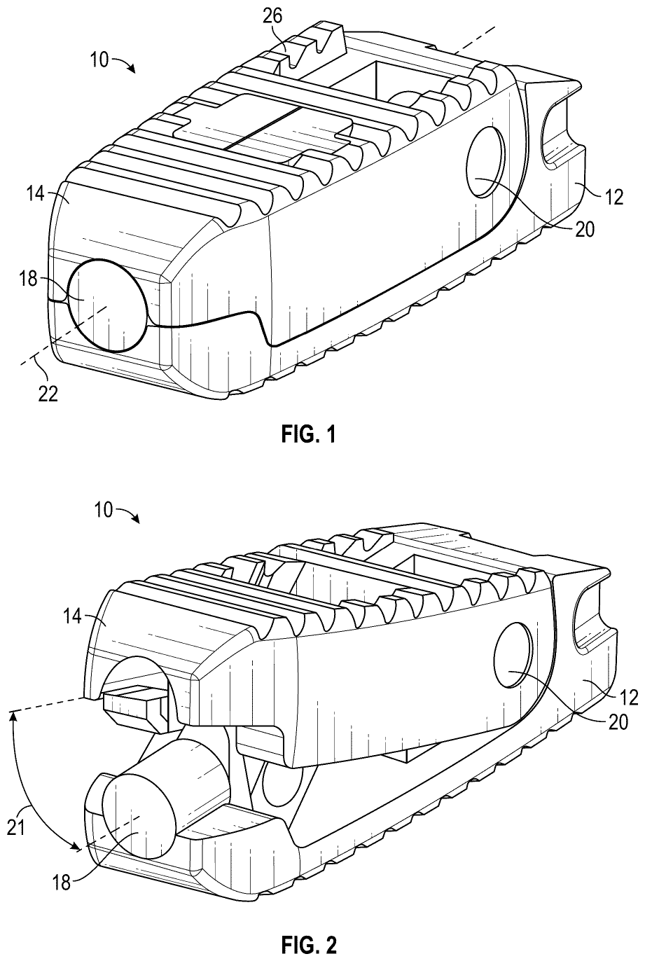

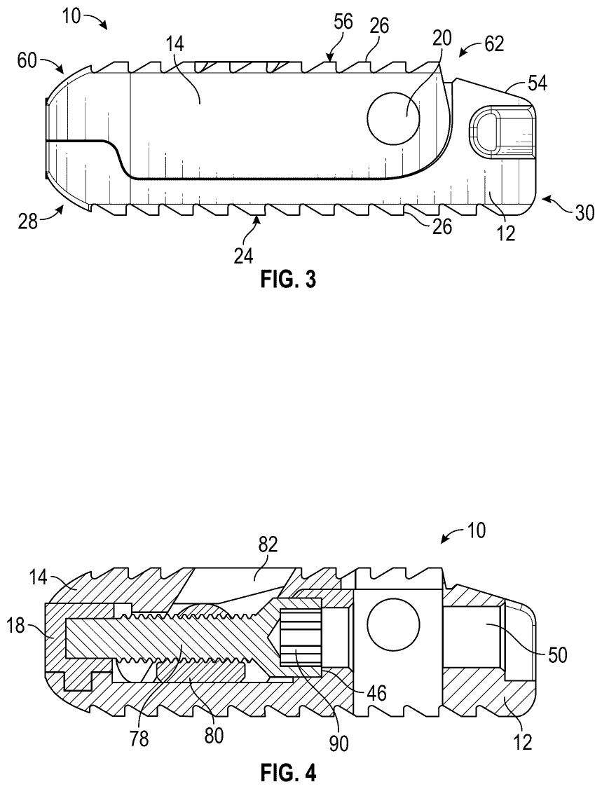

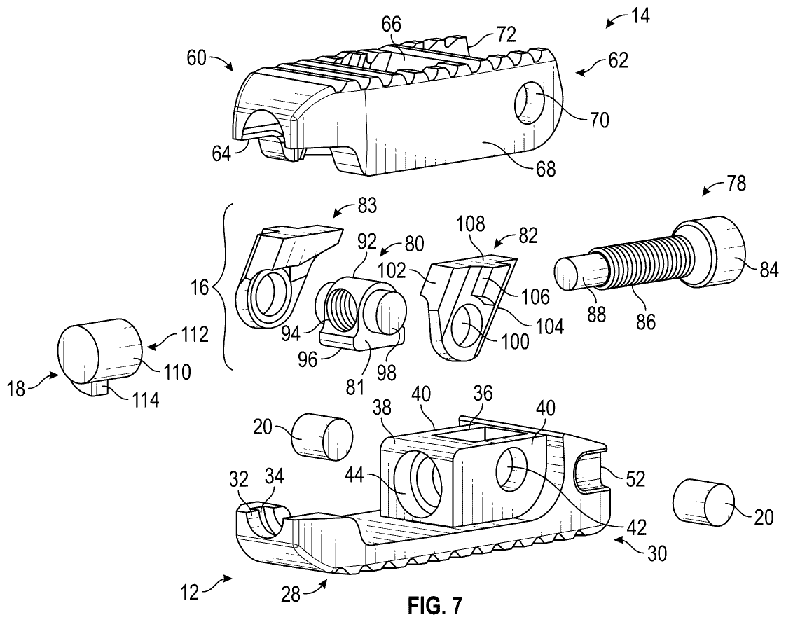

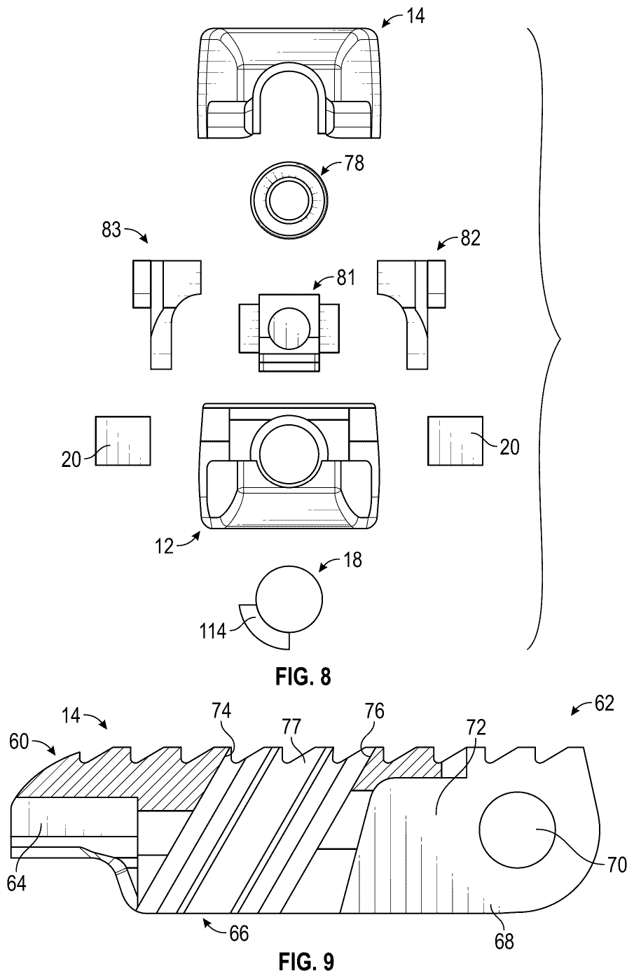

Referring now to FIGS. 1-8, an expandable implant 10 is shown according to one embodiment. Implant 10 is usable, for example, between and/or within portions of bone (e.g., between and/or within vertebral bodies or the spine or other portions of bone). In one embodiment, implant 10 includes a lower support 12 (e.g., a base support or assembly, a foundational plate, endplate, or member, etc.) and an upper support 14 (e.g., an adjustable support or assembly, a hinged plate, endplate, or member, etc.) adjustably coupled to the lower support 12 by way of a control assembly 16 (e.g., an adjustment assembly, etc.) and one or more pivot pins 20. In some embodiments, upper support 14 pivots relative to lower support 12 as a result of user manipulation of control assembly 16 (e.g., as a result of rotation or movement of a control shaft or member, etc.). In one embodiment, upper support 14 expands relative to lower support 12 in a lordotic fashion to mimic the natural curvature of the human spine. The amount of lordosis can be increased or decreased by manipulation of control assembly 16. An end cap 18 (e.g., a distal end member, etc.) assists in maintaining control assembly 16 in a desired position. Pivot pins 20 extend at least partially through lower support 12 and upper support 14 to enable relative pivoting adjustment between upper support 14 and lower support 12.

Implant 10 is movable between a collapsed position, as shown, for example, in FIGS. 1, 3, and 4, and an expanded position, as shown, for example, in FIGS. 2, 5, and 6. Further, implant 10 may be adjusted to any intermediate position between a fully collapsed position and a fully expanded position. Further yet, the amount of total expansion, (e.g., the maximum expansion angle 21 relative to axis 22 shown in FIGS. 1 and 2) may be varied to suit a particular application.

According to one embodiment, lower support 12 extends between a distal end 28 and a proximal end 30 and includes a bottom surface 24 having a plurality of ridges 26 (e.g., teeth, etc.) formed by corresponding grooves or channels. Ridges 26 are configured to facilitate gripping of adjacent portions of bone. A lower distal recess 32 is provided at distal end 28, and a retention groove 34 extends from lower distal recess 32. Retention groove 34 is configured to receive a retention projection 114 of end cap 18, as discussed in greater detail elsewhere herein. In some embodiments, lower support 12 includes an inner housing 36. Inner housing 36 is defined by a front wall 38 and side walls 40 that extend from front wall 38 toward proximal end 30 of lower support 12. Inner housing 36 in some embodiments defines a central aperture 48 (e.g., a cavity, etc.) providing access to an interior of implant 10. Central aperture 48 may be configured to receive bone growth material and/or bone material from adjacent portions of bone.

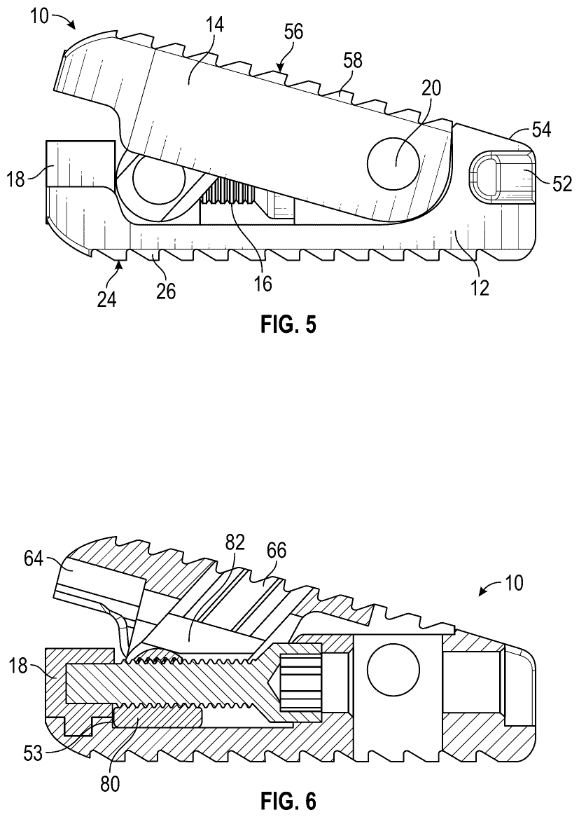

Lower support 12 further includes an access bore 50, tool recesses 52, and an inclined surface 54. Access bore 50 (see FIG. 4) provides access to central aperture 48 (e.g., for delivery of bone growth or other material) and control assembly 16 (e.g., to enable manipulation of control assembly 16 and control of the expansion and/or contraction of implant 10). Tool recesses 52 are configured to receive one or more tool portions to enable positioning of implant 10 in a desired position (e.g., within an intervertebral space, etc.). Inclined surface 54 (see FIG. 5) is in one embodiment configured such that when implant 10 is in an expanded configuration, inclined surface 54 is aligned with (e.g., substantially coplanar with) a top surface 56 of upper support 14 to provide additional support to adjacent portions of bone. In some embodiments, inclined surface 54 is angled downward in a proximal direction relative to a top surface 56 of upper support 14 when implant 10 is in a collapsed position. The angular position of inclined surface 54 is in some embodiments intended to accommodate the natural curvature of the human spine.

According to one embodiment, upper support 14 extends between a distal end 60 and a proximal end 62 and includes a top surface 56 having a plurality of ridges 58 (e.g., teeth, etc.) formed by corresponding grooves or channels. Ridges 58 are configured to facilitate gripping of adjacent portions of bone. An upper distal recess 64 is provided at distal end 60 and receives end cap 18. Sidewalls 68 extend downward relative to top surface 56.

In one embodiment, upper support 14 includes two opposing sidewalls 68. Each sidewall 68 includes a pivot pin aperture 70 configured to receive a pivot pin 20 there through to enable pivoting movement of upper support 14 relative to lower support 12. Upper support 14 also includes a rear aperture or cavity 72 that receives all or a portion of inner housing 36 when implant 10 is a collapsed position. A control aperture 66 extends through upper support 14 and is defined at least partially by distal ramp surfaces 74 and proximal ramp surfaces 76. An alignment channel 77 extends along each sidewall 68 and along control aperture 66. As discussed in further detail below, control aperture 66 receives portions of control assembly 16, and the angle of control aperture 66 relative to axis 22 may be designed to provide a desired rate of pivoting of upper support 14 relative to lower support 12.

In one embodiment, control assembly 16 includes a control shaft 78, a control member 80, and one or more pivot members 82. In some embodiments, control assembly 16 includes a pair of pivot members 82, 83 positioned on opposite sides of control member 80. Control shaft 78 is rotatable or otherwise manipulatable to cause translation or movement of control member 80 along control shaft 78. As control member 80 moves along control shaft 78, pivot members 82 move within control aperture 66 (see FIG. 6) to change the angular position of upper support 14 relative to lower support 12.

Control shaft 78 includes a head 84, a threaded portion 86, an end portion 88 and a receiver 90 provided in head 84. Head 84 defines a first end of control shaft 78 and end portion 88 defines a second opposite end of control shaft 78, with threaded portion 86 provided there between. Head 84 is received in a control member bore 44 and engages a shoulder 46 to limit proximal movement of control shaft 78 during use of implant 10. End portion 88 is received by end cap 18 to limit distal movement of control shaft 78.

Control member 80 is received on control shaft 78. In one embodiment, control member 80 includes a base member 81 and one or more pivot members 82. In some embodiments, control member 80 includes first and second pivot members 82, 83 pivotally coupled to opposite sides of base member 81.

Base member 81 includes a central portion 92 having a threaded bore 94 that threadingly engages threaded portion 86 of control shaft 78. Base member 81 further includes a bottom 96 and a pair cylindrical pivot bosses 98. Due to the threaded engagement of base member 81 onto control shaft 78, rotation of control shaft 78 causes movement (e.g., translational movement) of base member 81 along control shaft 78.

In one embodiment, each pivot member 82, 83 includes a pivot aperture 100 that receives one of the pivot bosses 98 to enable pivoting movement of pivot members 82, 83 relative to base member 81 about pivot bosses 98. Pivot member 82, 83 are mirror images of each other in one embodiment, and as such, pivot member 82 will be described in detail, with the understanding that pivot member 83 shares similar features. For example, pivot member 83 may include an alignment guide 107 that is similar to alignment guide 106.

Pivot member 82 includes distal ramp surface 102, proximal ramp surface 104, alignment guide 106, and top surface 108. Distal ramp surface 102 of pivot member 82 slidingly engages distal ramp surface 74 of upper support 14. Similarly, proximal ramp surface 104 of pivot member 82 slidingly engages proximal ramp surface 76 of upper support 14. During movement of base member 81 along control shaft 78, pivot members 82, 83 pivot about pivot bosses 98 as the corresponding distal and proximal ramp surfaces of the pivot members 82, 83 and upper support 14 engage, causing upper support 14 to move relative to lower support 12, and implant 10 to move toward an expanded or collapsed position, depending on the direction of rotation of control shaft 78.

Alignment guide 106 of pivot member 82 is received within alignment channel 77 of upper support 14 to maintain proper alignment between components and facilitate movement of upper support 14 relative to lower support 12. In some embodiments, when implant 10 is in a collapsed position, top surface 108 of pivot member 82 is generally aligned with top surface 56 of upper support 14. In some embodiments, top surface 108 may be substantially smooth, while in other embodiments, top surface 108 may be textured, include teeth or groves, or have other surface features.

End cap 18 includes a main body 110, a control shaft bore 112, and a retention projection 114. Control shaft bore 112 receives end portion 88 of control shaft 78. Retention projection 114 is received in retention groove 34 in lower support 12 to retain end cap 18 in place. In one embodiment, end cap 18 is rotated approximately 90 degrees to properly seat retention projection 114 within retention groove 34.

According to one embodiment, during use, a user positions implant 10 into a desired position, such as an intervertebral space, while collapsed, as shown, for example, in FIG. 1. To reposition implant 10, an appropriate tool may engage tool recesses 52 on lower support 12. In some embodiments, implant 10 is inserted into a space distal end first, with the appropriate tool engaging the proximal end of implant 10.

If desired, implant 10 may then be expanded to provide, for example, a desired amount of lordosis. Implant 10 may be expanded to a fully expanded position, or any intermediate expanded position between the fully collapsed position and the fully expanded position. In order to expand implant 10, in some embodiments, a user inserts an appropriate expansion tool through access bore 50 in lower support 12 and into receiver 90 in head 84 of control shaft 78. The expansion tool may then be used to manipulate the control shaft 78 to cause expansion of the implant 10. For example, receiver 90 may be hexagonal shaped, and the tool may be a hexagonal driver. Other suitable receivers and tools may be used according to various alternative embodiments.

As control shaft 78 is rotated, control member 80 translates along control shaft 78. For example, in one embodiment, to expand implant 10, control member 80 moves toward the distal end of lower support 12 as shown in FIGS. 4 and 6. Bottom 96 of base member 80 rides along a surface of lower support 12, and the travel of control member 80 is limited by limit shoulder 53, as shown in FIG. 6. In some embodiments, shoulder 53 is integrally formed (e.g., molded, etc.) with a remaining portion of lower support 12 to provide sufficient support for control shaft 78 during expansion of implant 10.

As control member 80 moves along control shaft 78, ramp surfaces on pivot members 82, 83 engage ramp surfaces of upper support 14 and cause upper support 14 to rotate about pivot pins 20. As upper support 14 pivots relative to lower support 12, pivot members 82 pivot about pivot bosses 98 on base member 81 to maintain proper alignment between the ramp surfaces on pivot members 82, 83 and the ramp surfaces on upper support 14.

In some embodiments and as shown in the FIGURES, the pivoting features of upper support 14 and pivot members 82, 83 maintain a generally parallel relationship between ramp surfaces 74, 76 of upper support 14 and ramp surfaces 102, 104 of pivot members 82, 83, which may facilitate the wedging action required to move upper support 14 relative to lower support 12.

If it is desirable to move implant 10 toward the collapsed position, control shaft 78 is rotated in an opposite direction from that used during expansion of implant 10. In one embodiment, to collapse implant 10, control member 80 moves toward the proximal end of lower support as shown in FIGS. 4 and 6. As control member 80 moves along control shaft 78, ramp surfaces on pivot members engage ramp surfaces of upper support 14 and cause upper support 14 to rotate about pivot pins 20.

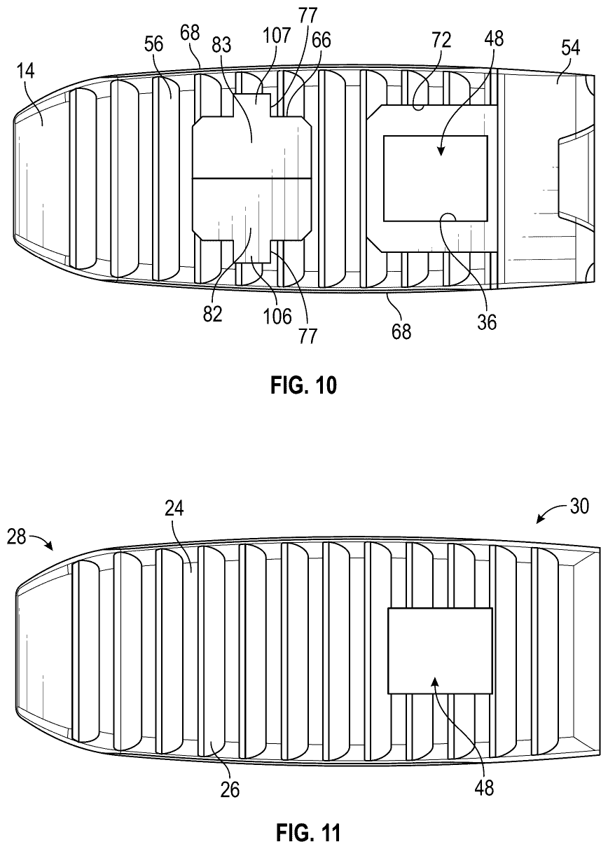

Referring now to FIG. 9, a cross-section view of the upper support 14 is shown according to an example embodiment. As shown, the upper support 14 includes a control aperture 66 configured to receive the pivot member 83. For example, the alignment guide 107 of the pivot member 83 may slide within the alignment channel 77 of the control aperture 66 as the implant 10 expands. Further, the distal ramp surface 74 and the proximal ramp surface 76 may interface with the ramp surfaces of the pivot member 83 when the implant 10 expands. The angle of expansion (e.g., angle 21) and the rate of angular expansion may be customized by altering the angles of the ramp surfaces 74, 76 and the alignment channel 77. It should be appreciated that the upper support 14 may also include a second control aperture 77 opposite the control aperture 66 configured to receive the pivot member 82 in a similar manner (see FIG. 10). The upper support 14 is also shown include a pivot pin aperture 70 configured to receive a pivot pin 20 there through to enable pivoting movement of upper support 14 relative to lower support 12.

Referring now to FIGS. 10 and 11, a top view and a bottom view, respectively, of the implant 10 is shown according to an example embodiment. As shown, pivot member 83 includes an alignment guide 107 that is received by the first alignment channel 77 in the upper support 14. Further, pivot member 82 includes an alignment guide 106 that is received by the second alignment channel 77 in the upper support. Further, as shown, the rear aperture 72 of the upper support 14 also receives all or a portion of the inner housing 36. The inner housing 36 further defines the central aperture 48 (e.g., a cavity, etc.) providing access to an interior of implant 10 from the top surface 56 of the upper support and from the bottom surface 24 of the lower support 12. Central aperture 48 may be configured to receive bone growth material and/or bone material from adjacent portions of bone.

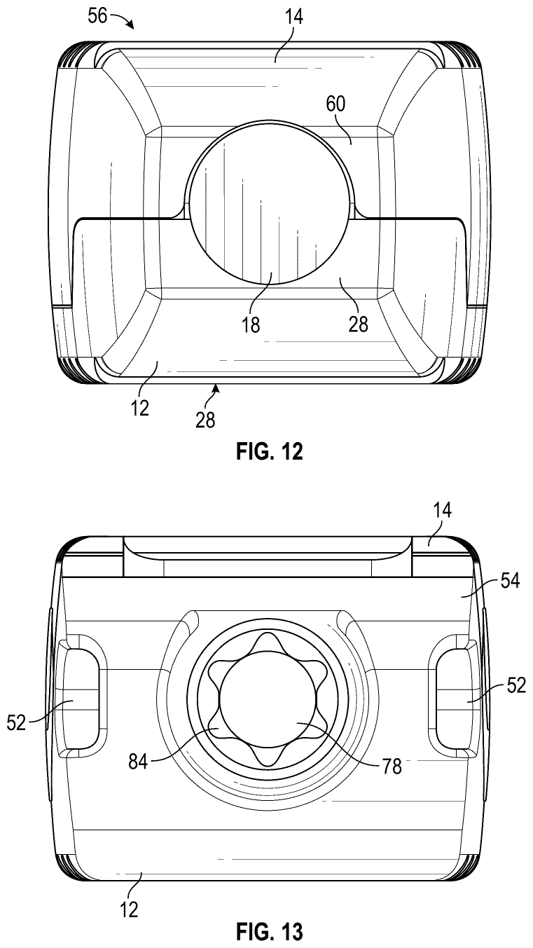

Referring now to FIGS. 12 and 13, a front and rear view, respectively, of the implant 10 are shown. As shown, in the collapsed position, the upper support 14 and the lower support 12 form a bull shaped nose that receives the end cap 18 at the front of the implant 10. The bull shaped nose all the implant 10 to be inserted into a desired location before the implant 10 is expanded. As shown in FIG. 13, the control shaft 78 is received by the rear of the implant 10. However, in operation, the head 84 is positioned away from the rear end of the implant 10, and is at least partially received by the control member bore 44 in the lower support 12

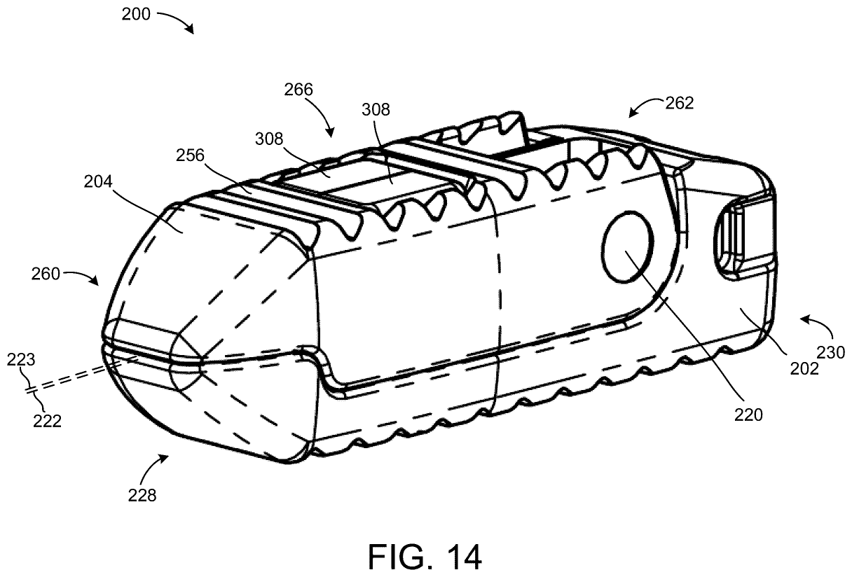

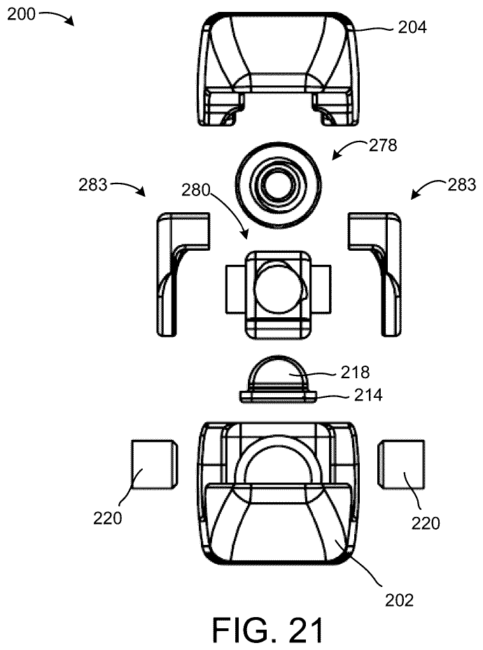

Referring generally to FIGS. 14-26, an implant 200 is shown, according to one embodiment. In an exemplary embodiment, the implant 200 of FIGS. 14-26 includes similar and/or the same features as the implant 10 of FIGS. 1-13. In this sense, similar reference numerals and/or naming conventions may be used to describe components that have the same or similar features.

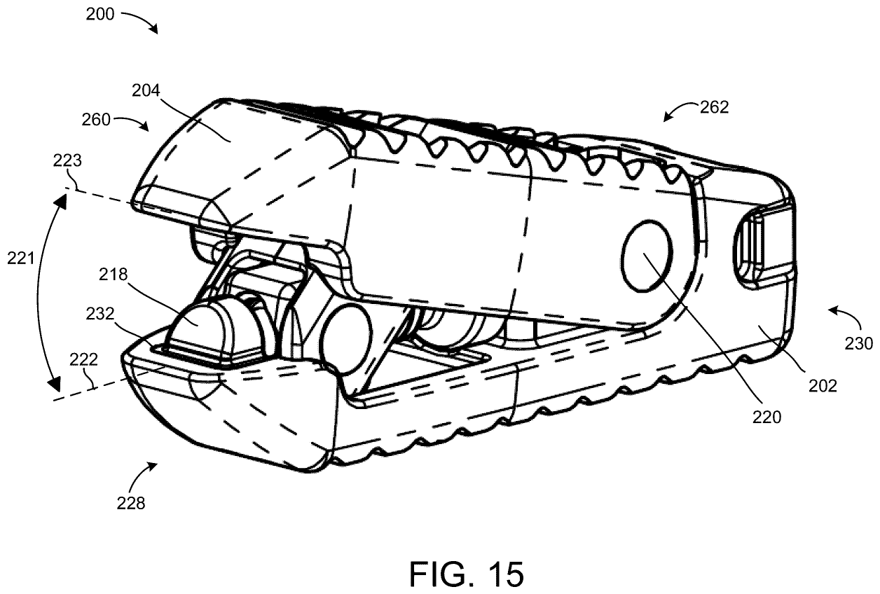

In an exemplary embodiment, the implant 200 is usable, for example, between and/or within portions of bone (e.g., vertebral bodies, the spine, other portions of bone, etc.). As shown, the implant 200 includes a lower support or base support, shown as a lower support 202, and an upper support or adjustable support, shown as upper support 204. The upper support 204 is adjustable coupled to the lower support 202, for example by way of a control assembly 206 (e.g., an adjustment assembly, etc.). As shown in at least FIG. 15, a cap 218 may be received by at least the lower support 202, for example to maintain a position and/or orientation of the control assembly 206 relative to the lower support 202. Further, at least one pivot pin, shown as pins 220, may extend at least partially through the lower support 202 and/or the upper support 204, for example to enable relative adjustment between the upper support 204 and the lower support 202. In an exemplary embodiment, the upper support 204 pivots relative to the lower support 202, for example as a result of manipulation of the control assembly 206 (e.g., as a result of rotation or movement of a control shaft or member, etc.). For example, the upper support 204 may expand relative to the lower support 202 in a lordotic fashion to mimic the natural curvature of the human spine. The amount of expansion (e.g., movement, etc.) can be increased or decreased by manipulation of control assembly 206.

As shown, the implant 200 is movable between a collapsed position (e.g., as shown in at least FIGS. 14 and 16-17) and an expanded position (e.g., as shown in at least FIGS. 15 and 18-19). For example, the implant 200 may be movable between a fully collapsed position (e.g., as shown in at least FIG. 14), and an expanded position (e.g., as shown in at least FIG. 15). As shown, in the fully collapsed position, an axis and/or plane of the lower support 202 (e.g., shown as axis 222) may be aligned with (e.g., parallel to, aligned with, etc.) an axis and/or plane of the upper support 204 (shown as axis 222). Further, the upper support 204 is movable relative to the lower support 202, for example such that the upper support 204 and the lower support 202 form an angle 221 therebetween (e.g., between the axis 222 and the axis 223, etc.).

It should be understood that while the lower support 202 and the upper support 204 are described herein as defining one or more axis (e.g., the axis 222, the axis 223, etc.), it is contemplated that the lower support 202 and/or the upper support 204 may similarly define one or more planes (e.g., defined by the axis, etc.) that may also be movable relative to one another. Further, it should be understood that the implant 200 (e.g., the angle 221) may be adjusted to any intermediate position between a fully collapsed position and a fully expanded position (e.g., 15, 25, 30, 45, 50, 60, etc. degrees). Yet further, it should be understood that the amount of expansion (e.g., the angle 221 shown in FIGS. 14-15) may be varied, for example to suit a particular application. All such embodiments and variations are contemplated herein.

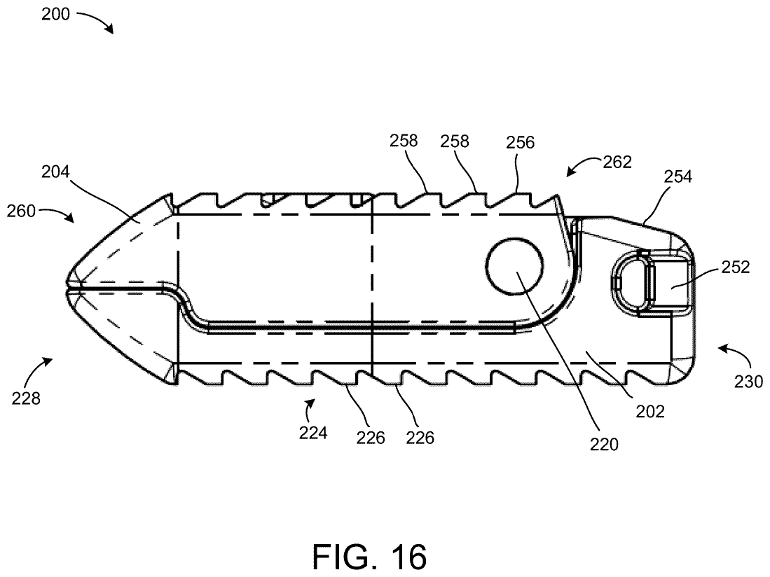

As shown in at least FIG. 16, the lower support 202 extends between a first end, shown as distal end 228, and a second end, shown as proximal end 230. The lower support 202 is shown to include a bottom surface 224 having a plurality of textured surfaces or interfaces, shown as ridges 226, which are formed by corresponding grooves and channels. In some embodiments, the bottom surface 224 includes additional surfaces or interfaces (e.g., teeth, serrations, etc.), for example to facilitate securing (e.g., gripping, etc.) adjacent portions of bone.

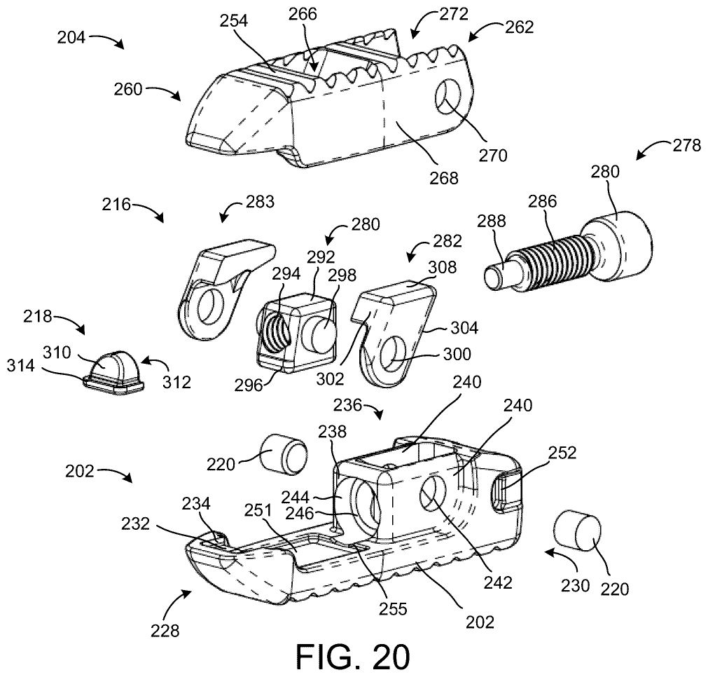

As shown in FIG. 20, the distal end 228 includes a lower distal recess 232, which partially extends into the lower support 202. As shown, the lower distal recess 232 extends into the lower support 202 (e.g., toward the bottom surface 224, etc.), for example to form an opening or aperture within the lower support 202 to receive the cap 218. In an exemplary embodiment, the lower distal recess 232 is offset from an end or tip of the lower support 202 (e.g., offset from an end of the distal end 228, toward the proximal end 230, etc.). As shown in at least FIG. 15, the lower distal recess 232 may be centered relative to the lower support 202 (e.g., along the axis 222, etc.). In an exemplary embodiment, the lower distal recess 232 includes a retention guide or groove, shown as retention groove 234 (e.g., in at least FIG. 20). The retention groove 234 may extend around an exterior or external portion of the lower distal recess 232, and is configured to receive a retention flange, projection or protrusion, shown as retention projection 314, of the cap 218, for example to position align, and/or retain the cap 218 relative to the lower support 202. In other embodiments, the lower distal recess 232 is otherwise arranged and/or configured.

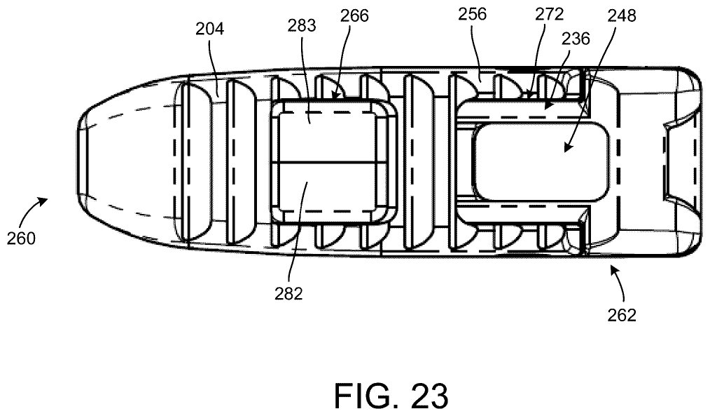

As also shown in at least FIG. 20, the lower support 202 further includes a housing or inner structure, shown as inner housing 236. The inner housing 236 may be defined by a front wall 238, and one or more sidewalls, shown as sidewalls 240, that extend from the front wall 238 toward the proximal end 230 of the lower support 202. In an exemplary embodiment, the inner housing 236 defines a central cavity or aperture, shown as central aperture 248 in at least FIG. 23, or example to provide access to an interior of the implant 200. For example, the central aperture 248 may be configured to receive bone growth material and/or bone material (e.g., from adjacent portions of bone, etc.), for example to stabilize the lower support 202 relative to adjacent portions of bone.

As shown in at least FIG. 20, each sidewall 240 also includes an aperture or opening, shown as pivot pin aperture 242, which may be configured to receive a pivot pin (e.g., the pivot pin 20, etc.), for example to enable pivoting of the upper support 204 relative to the lower support 202. Further, the front wall 238 is shown to include an aperture or opening, shown as control member bore 244, which extends through the front wall 238 toward the proximal end 230 of the lower support 202. The control member bore 244 is also shown to include at least one collar, projection, protrusion, ridge, shoulder, or limit, shown as collar 246 (e.g., extending around at least a portion of the control member bore 244, etc.). In an exemplary embodiment, and as will be described herein, the control member bore 244 (and/or the collar 246, etc.) may receive and/or engage a control member, for example to position the control member relative to the lower support 202 and/or to limit movement of the control member (e.g., proximal movement, etc.) during use of the implant 200.

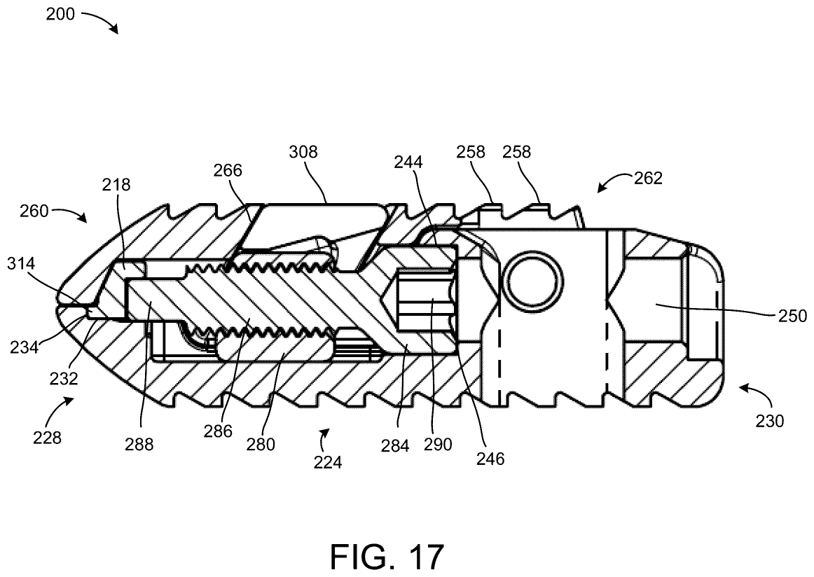

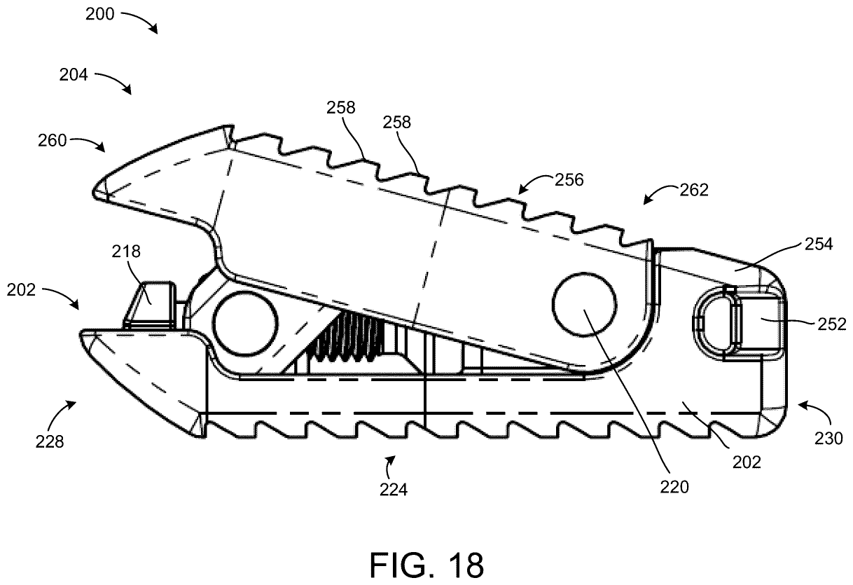

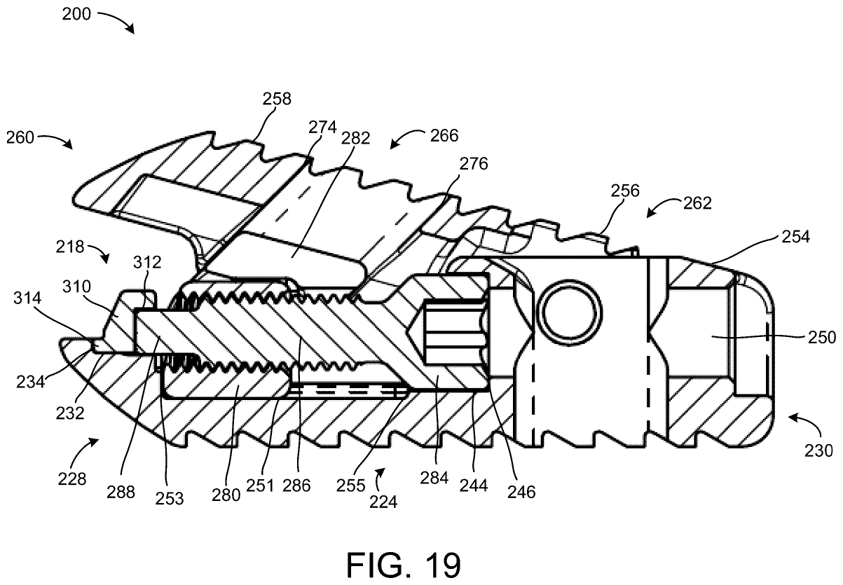

As shown in at least FIGS. 17-18, the lower support 202 further includes an access aperture or bore, shown as access bore 250, at least one tool recess, shown as tool recesses 252, and a surface 254. In an exemplary embodiment, the access bore 250 provides access to the central aperture 248 (e.g., for delivery of bone growth material or other material, etc.). Further, the access bore 250 also provides access to the control assembly 216, for example to enable manipulation of the control assembly 216 to control expansion and/or contraction of the implant 200. The tool recesses 252 may be configured to receive one or more tool portions, for example to enable positioning of the implant 200 in a predetermined or desired position (e.g., within the intervertebral space, etc.). Further, the surface 254 may be an inclined surface such that when the implant 200 is in an expanded configuration (e.g., a pivot configuration, etc.), the surface 254 aligns with (e.g., is coplanar with, substantially coplanar with, etc.) a top surface 256 of the upper support 204 (e.g., as shown in at least FIG. 19). In this regard, the surface 254 may be configured to provide additional support to adjacent bones or bone portions. In some embodiments, the surface 254 is otherwise angled and/or oriented, for example at a downward angle relative to the top surface 256 of the upper support 204 when the implant is in a collapsed position (e.g., as shown in at least FIG. 16).

Referring generally to FIGS. 16-20, the upper support 204 extends between a first end, shown as distal end 260, and a second end, shown as proximal end 262. Like the lower support 202, the upper support 204 is also shown to include a surface, shown as the top surface 256, having a plurality of textured surfaces or interfaces, shown as ridges 258, which are formed by corresponding grooves and channels. In some embodiments, the top surface 256 includes additional surfaces or interfaces (e.g., teeth, serrations, etc.), for example to facilitate securing (e.g., gripping, etc.) adjacent portions of bone.

In an exemplary embodiment, the upper support 204 includes at least one sidewall. For example, the upper support 204 is shown to include two opposing sidewalls, shown as sidewalls 268, where at least a portion of the top surface 256 extends between the sidewalls 268. As shown, each sidewall 268 includes an aperture or opening, shown as pivot pin aperture 270, which may be configured to receive a pivot pin (e.g., the pivot pin 20, etc.), for example to enable pivoting of the upper support 204 relative to the lower support 202.

As shown in at least FIGS. 20-23, the upper support 204 also includes one or more apertures or cavities. For example, the upper support 204 may include a first or control aperture, shown as control aperture 266, and a second or rear cavity or aperture, shown as rear cavity 272. In an exemplary embodiment, the rear cavity 272 is configured to receive all or a portion of the inner housing 236, for example when the implant 200 is in a collapsed position.

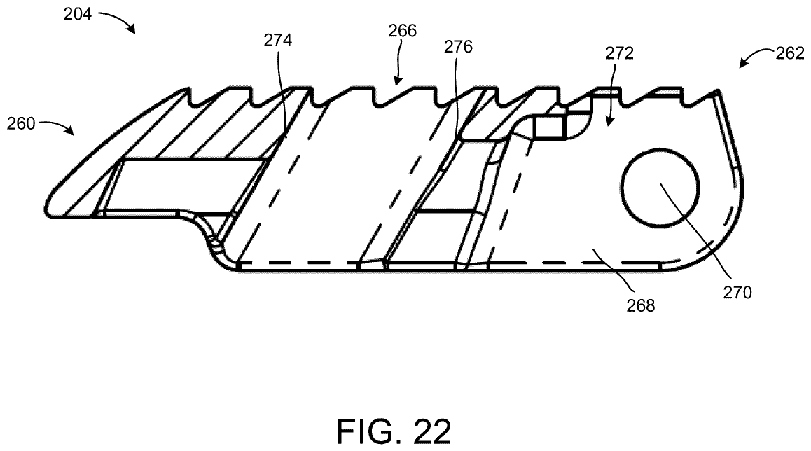

According to an exemplary embodiment, the control aperture 266 extends through the upper support 204 (e.g., the top surface 256, etc.), for example to define (or be defined by) one or more surfaces or interfaces. For example, and as shown in at least FIG. 22, the control aperture 266 extends through the upper support 204, and is defined by a first ramp or surface, shown as distal ramp surface 274, and a second ramp or surface, shown as proximal ramp surface 276. As will be discussed herein, the control aperture 266 may be configured to receive one or more portions of the control assembly 216, and the configuration of the control aperture 266 (e.g., an angle of the ramp surfaces 274, 276, etc. relative to the axis 222, etc.) may be designed to control a rate of movement (e.g., pivoting, expansion, etc.) of the upper support 204 relative to the lower support 202. In some embodiments, the control aperture 266 further includes or defines one or more alignment components (e.g., one or more alignment channels, etc.), for example to maintain an alignment of components of the control assembly 216 relative to the upper support 204 (e.g., within the control aperture 266, etc.) during expansion and/or contraction of the implant 200.

Referring back to FIG. 20, the control assembly 216 is shown to include a drive member or control shaft, shown as control shaft 278, at least one control member, shown as control member 280, and at least one pivot member. In an exemplary embodiment, the control assembly 216 includes a pair of pivot members, shown as pivot members 282, 283, for example on opposite sides of the control member 280. The control shaft 278 may be received by the control member 280 and/or manipulated (e.g., rotated, otherwise manipulatable, etc.), for example to cause movement (e.g., translation, etc.) of the control member 280 along the control shaft 278. According to an exemplary embodiment, as the control member 280 moves relative to the control shaft 278, the pivot members (e.g., pivot members 282, 283, etc.) may move within the control aperture 266 (e.g., as shown in at least FIG. 19), for example to control a position of the upper support 204 relative to the lower support 202 (e.g., an angular position or orientation, etc.).

As shown in FIG. 20, the control shaft 278 includes a head 284, a threaded portion 286, and an end portion 288. In an exemplary embodiment, the head 284 defines a first end of the control shaft 278, and the end portion 288 defines a second end, opposite the first end, of the control shaft 278. The threaded portion 286 is shown to be provided therebetween. As shown in at least FIG. 17, the head 284 also includes a receiver 290, for example to receive (e.g., couple, etc.) a tool (e.g., an expansion tool, a driver, etc.) and/or to transmit a force to the control shaft 278.

As shown in FIGS. 17 and 19, in an exemplary embodiment the head 284 is received in the control member bore 244 (e.g., of the inner housing 236 of the lower support 202, etc.). Further, the head 284 may be configured to engage the collar 246, for example to control movement of the control shaft 278 (e.g., limit proximal movement, etc.) during use of the implant 200 (e.g., as shown in at least FIG. 19). In an exemplary embodiment, the end portion 288 is received by the cap 218, for example to position the control shaft 278 relative to the cap 218 and/or the lower support 202. For example, and as described herein, the cap 218 may be received by the lower distal recess 232 of the lower support 202 (e.g., via the engagement between the retention projection 314 of the cap 218 and the retention groove 234 of the lower distal recess 232, etc.). With the cap 218 positioned relative to the lower support 202, the cap 218 and/or the lower support 202 (e.g., the lower distal recess 232, etc.) may receive the end portion 288 of the control shaft 278 (e.g., as shown in at least FIG. 17), for example to position the control shaft 278 relative to the cap 218 and/or the lower support 202.

Referring back to FIG. 20, the control member 280 may be or include a base member, which is shown to include a central or body portion, shown as central portion 292, having a bore, shown as bore 294, extending through at least a portion of the central portion 292. In an exemplary embodiment, the central portion 292 is substantially square or cubical, and/or the bore 294 is a threaded bore, for example to engage (e.g., receive, interface with, etc.) the threaded portion 286 of the control shaft 278. The engagement between the bore 294 and the control shaft 278 (e.g., the threaded engagement, etc.) may be such that manipulation of the control shaft 278 (e.g., rotation of the control shaft 278, etc.) causes relative movement (e.g., translation movement, etc.) of the control member 280 relative to the control shaft 278 (e.g., along the control shaft 278, etc.).

As also shown in FIG. 20, the control member 280 further includes a base or bottom, shown as bottom 296, and at least one boss or projection, shown as pivot boss 298. In an exemplary embodiment, the bottom 296 is substantially square or rectangular, and may be configured to engage (e.g., interface, movably couple, etc.) a surface of the lower support 202 (e.g., a recess or recess surface, etc.). Further, the control member 280 may include a pair of pivot bosses 298 extending from opposing sides of the control member 280, which may be configured to movably couple (e.g., rotatably couple, etc.) pivot members (e.g., the pivot members 282, 283, etc.). It should be understood that in other embodiments, the components of the control member 280 (e.g., the central portion 292, the bore 294, the bottom 296, the pivot bosses 298, etc.) may be otherwise arranged and/or configured. For example, in some embodiments, the control member 280 includes a single pivot boss 298.

Referring still to FIG. 20, the pivot member are shown to include a pivot aperture, for example to enable pivoting movement of the pivot member relative to the control member 280. In an exemplary embodiment, each pivot member 282, 283 includes a pivot aperture 300, which receives one of the pivot bosses 298, for example to enable pivoting movement of the pivot members 282, 283 relative to the control member 280 (e.g., about the pivot bosses 298, etc.). In some embodiments, the pivot members 282, 283 include the same or similar components. In this regard, while the pivot member 282 is described in detail herein, it should be understood that the pivot member 283 may share the same or similar features.

As shown, the pivot member 282 includes one or more ramps or surfaces. For example, the pivot member 282 is shown to include a first or distal ramp or surface, shown as distal ramp surface 302, a second or proximal ramp or surface, shown as proximal ramp surface 304, and a third or top surface, shown as top surface 308. In an exemplary embodiment, the distal ramp surface 302 of the pivot member 282 engages (e.g., slidable engages, interfaces, etc.) the distal ramp surface 274 of the upper support 204 (e.g., within the control aperture 266). Similarly, the proximal ramp surface 304 of the pivot member 282 engages (e.g., slidably engages, interfaces, etc.) the proximal ramp surface 276 of the upper support 204 (e.g., within the control aperture 266).

As an exemplary illustrative example, in response to manipulation of the control shaft 278, the control member 280 moves along the control shaft 278. As the control member 280 moves, the pivot members 282, 283 pivot about the pivot bosses 298 of the control member 280, for example causing corresponding distal and proximal ramp surfaces of the pivot members 282, 283 and the upper support 204 (e.g., within the control aperture 266, etc.) to engage (e.g., interface, slidable engage, etc.). The engagement between the ramp surfaces cause the upper support 204 to move relative to the lower support 202 (e.g., pivot, etc.), thereby causing the implant 200 to move toward and expanded or collapsed position (e.g., depending on a direction of rotation of the control shaft 278 and/or movement of the control member 280, etc.).

According to an exemplary embodiment, and as shown in at least FIG. 14, when the implant 200 is in a collapsed position, the top surface 208 of the pivot member (e.g., the pivot members 282, 283, etc.) is generally aligned (e.g., parallel to, etc.) with the top surface 256 of the upper support 204 (e.g., within the control aperture 266, etc.). In other embodiments, the control member 280 and/or the pivot members 282, 283 include additional alignment components. For example, the pivot members 282, 283 may include alignment projections or protrusions, for example to control movement of the pivot members 282, 283 relative to the upper support 204 (e.g., within the control aperture 266, etc.).

Referring back to FIG. 20, the cap 218 is shown to include a main body or base, shown as main body 310, a control shaft or bore, shown as bore 312, and the retention projection 314. In an exemplary embodiment, and as described elsewhere herein, the bore 312 is configured to receive the end portion 288 of the control shaft 278, and the retention projection 314 is received within the retention groove 234 of the lower support 202, for example to position (e.g., retain, maintain, etc.) the cap 218 relative to the lower support 202 and/or the control shaft 278 relative to the cap 218 and/or the lower support 202. In an exemplary embodiment, the square or rectangular configuration of the main body 310 and/or the retention projection 314 (e.g., and corresponding portions of the lower support 202, etc.) are configured such that the cap 218 is movable relative to and/or within the lower support 202 (e.g., along the axis 222, etc.), for example to seat the retention projection 314 within the retention groove 234.

Referring still to FIG. 20, the lower support 202 is shown to further include a recess, guide, or control, shown as recess 251. According to an exemplary embodiment, the recess 251 extends into a bottom portion or surface of the lower support 202 (e.g., toward the bottom surface 224, etc.), for example forming a void, space, or opening at a bottom portion or surface of the lower support 202. As shown in at least FIGS. 19-20, the recess 251 may include and/or be defined by one or more boundaries, ridges, projections, protrusions, shoulders, or limits. For example, the recess 251 is shown to be defined by a peripheral boundary or wall, shown as a first limit 253 (e.g., a first portion of the peripheral wall defining the recess and positioned toward the distal end 228, etc.), and a peripheral boundary or wall, shown as a second limit 255 (e.g., a second portion of the peripheral wall defining the recess positioned toward the proximal end 230, etc.).

According to an exemplary embodiment, and as will be described herein, the recess 251 may be configured to receive a portion of the control assembly 216, for example to limit, control and/or guide movement of the control assembly 216 relative to the lower support 202. For example, the recess 251 may receive the control member 280 (e.g., interface with the bottom 296, etc.). As the control shaft 278 is manipulated, the control member 280 (e.g., the bottom 296, etc.) may move relative to the recess 251 (e.g., translate along, move within, etc.). Further, the movement of the control member 280 may be controlled (e.g., limited, etc.), for example by an engagement with the first limit 253 (e.g., as shown in at least FIG. 19) and/or the second limit 255, for example to control an amount of expansion and/or contraction of the implant 200. In some embodiments, the recess 251 is integrally formed (e.g., molded, machined, etc.) within a portion of the lower support 202, for example to allow for the lower support 202 to maintain sufficient integrity during expansion/contraction of the implant 200, while allowing one or more surfaces defining the recess 251 to support the control assembly 216 during expansion/contraction of the implant 200.

Referring generally to FIGS. 22-26, various components of the implant 200 are shown in greater detail, according to an example embodiment. For example, and as shown in FIG. 22, a cross-section view of the upper support 204 is shown, according to an example embodiment. As shown, and as described elsewhere herein, the upper support 204 includes the control aperture 266, which may be configured to movably receive the pivot members 282, 283. For example, the distal ramp surface 274 and the proximal ramp surface 276 of the upper support 204 may interface with (e.g., engage, slidably engage, etc.) the ramp surfaces 302, 304 of the pivot members 282, 283, for example to cause the implant 200 to expand and/or contract. The angle of expansion of the implant 200 (e.g., the angle 221 shown in FIG. 15) and/or the rate of expansion of the implant 200 may be altered and/or modified, for example by altering or modifying the angle of the ramp surfaces 274, 276 and/or ramp surfaces 302, 304. As shown, and as also described herein, the upper support 204 is shown to include the pivot pin aperture 270, for example to receive a pin 220 to enable movement (e.g., pivoting, etc.) of upper support 204 relative to lower support 202.

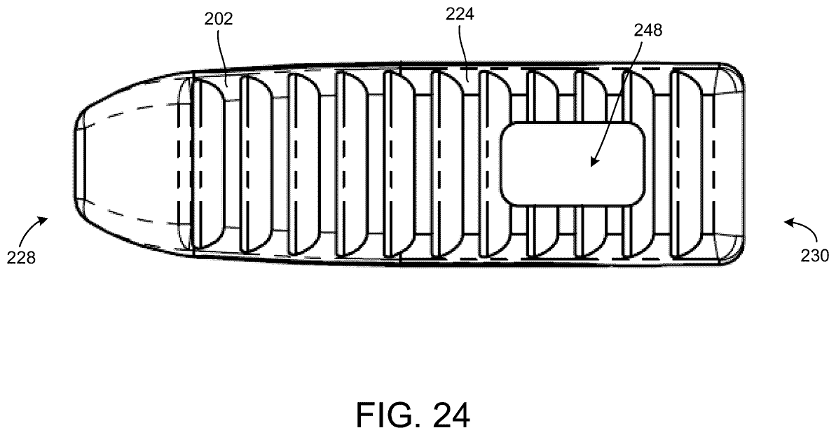

As shown in FIGS. 23-24, a top view and a bottom view, respectively, of the implant 200 are shown, according to an example embodiment. As shown, the pivot members 282, 283 are received in the control aperture 266 of the upper support 204 (e.g., as shown in FIG. 23). Further, the rear cavity 272 of the upper support 204 is shown to receive at least a portion of the inner housing 236 of the lower support 202. As shown, the inner housing 236 defines the central aperture 248, for example to provide access to an interior of the implant 200 (e.g., from the top surface 256 of the upper support 204 and/or the bottom surface 224 of the bottom support 202, etc.), for example to receive bone growth material and/or bone material from adjacent portions of bone.

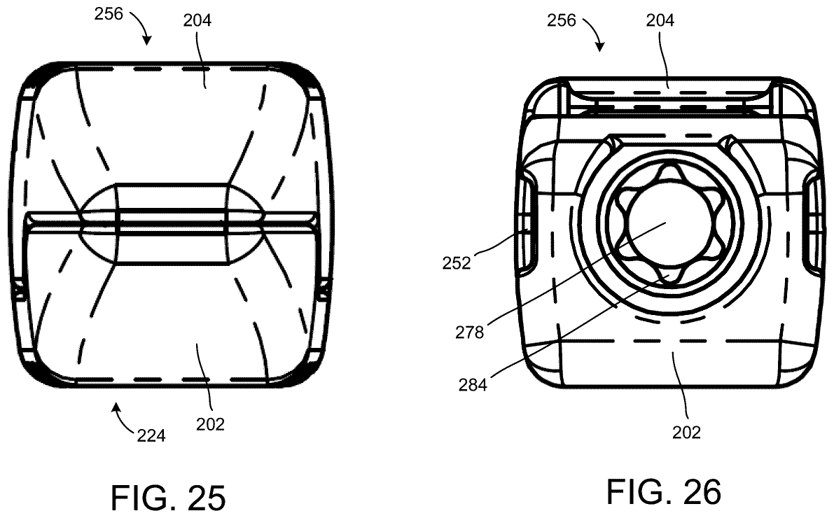

As shown in FIGS. 25-26, a front and rear view, respectively, of the implant 200 are shown. As shown, in the collapsed position, the upper support 204 and the lower support 202 form a bull shaped nose, which contains the cap 218 (not shown) at the front of the implant 200. In an exemplary embodiment, the shape of the front of the implant 200 (e.g., the bull shaped nose, etc.) allows the implant 200 to be inserted into a desired location, for example before the implant 200 is expanded. As shown in FIG. 26, the control shaft 278 is received by the rear of the implant 200 (e.g., the control member bore 244 of the inner housing 236 of the lower support 202, etc.). It should be understood that in operation, the head 284 of the control shaft 278 is positioned away from a rear end of the implant 200 (e.g., a proximal end of the implant 200, etc.), for example toward a central portion of the implant 200 (e.g., within the control member bore 244, etc.) as described herein.

As an illustrative example, during use, a user positions implant 200 into a desired position, such as an intervertebral space, while collapsed, as shown, for example, in FIG. 14. To reposition implant 200, an appropriate tool may engage tool recesses 252 on lower support 202. In some embodiments, implant 200 is inserted into a space distal end first (e.g., the end with the bull shaped nose, etc.), with the tool engaging the proximal end of implant 200 (e.g., the end with the tool recesses 252, etc.).

If desired, implant 200 may then be expanded to provide, for example, a desired amount of lordosis. In an exemplary embodiment, the implant 200 may be expanded to a fully expanded position, and/or any intermediate expanded position between the fully collapsed position and the fully expanded position. In order to expand implant 200, in some embodiments, a user or operator inserts an appropriate expansion tool through the access bore 250 in the lower support 202, and/or into the receiver 290 in the head 284 of control shaft 278. The tool may then be used to manipulate the control shaft 278, for example to cause expansion of the implant 200. For example, the receiver 290 may be hexagonal shaped, and the tool may be a hexagonal driver. It should be understood that other suitable receivers and/or tools may be used, according to various alternative embodiments.

As the control shaft 278 is manipulated (e.g., rotated, etc.), the control member 280 translates along the control shaft 278. For example, in one embodiment, to expand the implant 200, the manipulation of the control member 280 causes the control member 280 (e.g., via the threaded engagement, etc.) to move toward the distal end 228 of the lower support 202 (e.g., within the recess 251), for example as shown in at least FIGS. 17 and 19. As described herein, the bottom 296 of the control member 280 moves within the recess 251, for example along a bottom surface of the recess 251, and the travel of the control member 280 may be controlled (e.g., limited, etc.) by the first limit 253 (e.g., as shown in at least FIG. 19).

As the control member 280 moves along control shaft 278, the ramp surfaces on of one or more of the pivot members (e.g., the pivot members 282, 283, etc.) engage corresponding ramp surfaces of the upper support 204 (e.g., within the control aperture 266, etc.), and cause the upper support 204 to rotate about pins 220. Further, as the upper support 204 pivots relative to the lower support 202, the pivot members (e.g., the pivot members 282, 283, etc.) pivot about pivot bosses 298 on control member 280, for example to maintain an alignment between the ramp surfaces on the pivot members (e.g., pivot members 282, 283, etc.) and the ramp surfaces on the upper support 204 (e.g., within the control aperture 266, etc.).

In some embodiments, the pivoting features of the upper support 204 and/or the pivot members (e.g., the pivot members 282, 283, etc.) maintain a generally parallel relationship between the ramp surfaces 274, 276 of upper support 204 and the corresponding ramp surfaces 302, 304 of the pivot members (e.g., the pivot members 282, 283, etc.), which may facilitate the movement (e.g., pivoting action, etc.) between the upper support 204 relative and the lower support 202.

If it is desirable to move implant 200 toward the collapsed position, the control shaft 278 manipulated (e.g., rotated, etc.) in an opposite direction from that used during expansion of implant 200. In one embodiment, to collapse implant 200, the control member 280 moves toward a proximal end of lower support 202 (e.g., the proximal end 230 of the lower support 202, etc.). As the control member 280 moves along control shaft 278, the ramp surfaces of the pivot members engage the corresponding ramp surfaces of upper support 204, and cause upper support 204 to rotate about pins 220, as described elsewhere herein.

As utilized herein with respect to numerical ranges, the terms “approximately,” “about,” “substantially,” and similar terms generally mean+/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. All such variations are within the scope of the disclosure.

It is important to note that the construction and arrangement of the expandable implant as shown in the various exemplary embodiments is illustrative only. Additionally, any element disclosed in one embodiment may be incorporated or utilized with any other embodiment disclosed herein. It should be appreciated that elements of the various embodiments may be incorporated or utilized with any of the other embodiments disclosed herein.

What is claimed is:

1. An expandable implant comprising:
a first support comprising an exterior surface configured to engage bone and a control aperture, the control aperture defined by the first support and extending through the exterior surface;
a second support movably coupled to the first support, the second support comprising a recess defined by a peripheral wall; and
a control assembly comprising:
a control shaft;
a base member received on the control shaft, the base member configured to translate along the control shaft within the recess, wherein translational movement of the base member along the control shaft and within the recess is limited by a first portion of the peripheral wall; and
a pivot member pivotally coupled to the base member, the pivot member configured to move within the control aperture,
wherein translation of the base member along the control shaft causes the pivot member to pivot relative to the base member, and the first support to move relative to the second support to change an angle between the first support and the second support.

2. The expandable implant of claim 1, wherein the recess is further defined by second portion of the peripheral wall.

3. The expandable implant of claim 2, wherein translational movement of the base member along the control shaft within the recess in a first direction is limited by the first portion of the peripheral wall, and translational movement of the base member along the control shaft within the recess in a second direction is limited by the second portion of the peripheral wall.

4. The expandable implant of claim 1, wherein the peripheral wall is continuous about the recess.

5. The expandable implant of claim 1, wherein the control aperture defines a first ramp surface configured to slidably engage a second ramp surface on the pivot member.

6. The expandable implant of claim 1, wherein the pivot member comprises first and second pivot members independently coupled and rotatable to opposite sides of the base member.

7. The expandable implant of claim 4, wherein the first pivot member is received on a first boss on a first side of the base member and the second pivot member is received on a second boss on a second side of the base member.

8. The expandable implant of claim 1, further comprising:
a cap received by a recess at an end of the second support, wherein the cap is configured to rotatably receive an end portion of the control shaft to permit rotational movement of the control shaft and vertically fix the control shaft relative to the second support.

9. An expandable implant comprising:
a first support defining a control aperture extending through the first support;
a second support movably coupled to the first support, the second support comprising a first end having an access bore and a second end opposite the first end and comprising a recess;
a control assembly comprising:
a control shaft movably coupled with the second support, wherein the access bore provides tool access to the control shaft via the access bore;
a base member received on the control shaft; and
a pivot member pivotally coupled to the base member and movably coupled with the control aperture; and
a cap received by the recess, the cap configured to receive an end portion of the control shaft to position the control shaft relative to the second support,
wherein translation of the base member along the control shaft causes the pivot member to pivot relative to the base member, and the first support to move relative to the second support to change an angle between the first support and the second support.

10. The expandable implant of claim 9, wherein the cap is configured to rotatably receive the end portion of the control shaft to permit rotational movement of the control shaft and vertically fix the control shaft relative to the second support.

11. The expandable implant of claim 9, wherein the recess is offset from a tip of the second end of the second support.

12. The expandable implant of claim 9, wherein the recess comprises a retention groove and the base comprises a retention projection extending from the base, wherein the retention groove is configured to receive the retention projection to seat the cap within the recess.

13. The expandable implant of claim 9, wherein the recess of the second support is a lower recess, and wherein the first support further comprises an upper recess,
wherein when the expandable implant is in a collapsed position the cap is received by the lower recess and the upper recess.

14. The expandable implant of claim 9, wherein the second support further comprises a recess defined by a peripheral wall, and wherein translational movement of the base member along the control shaft and within the recess is limited by a first portion of the peripheral wall.

15. The expandable implant of claim 14, wherein the recess is further defined by a second portion of the peripheral wall, and wherein translational movement of the base member along the control shaft within the recess in a first direction is limited by the first portion of the peripheral wall, and translational movement of the base member along the control shaft within the recess in a second direction is limited by the second portion of the peripheral wall.

16. The expandable implant of claim 9, wherein the pivot member comprises first and second pivot members coupled to opposite sides of the base member.

17. The expandable implant of claim 9, wherein the second support comprises a housing defining the access aperture, a cavity, and a control member bore, wherein the control member bore receives a head of the control shaft.

18. An expandable implant comprising:
a first support defining a control aperture extending through the first support;
a second support movably coupled to the first support, the second support comprising a recess defined by a boundary wall; and
a control assembly comprising:
a control shaft movably coupled with the second support;
a base member movably received on the control shaft, wherein movement of the base member along the control shaft is limited by a first portion of the boundary wall; and
a pivot member pivotally coupled to the base member and movably coupled with the control aperture; and
a cap received by a lower recess of the second support, the cap configured to receive the control shaft to position the control shaft relative to the second support, and
wherein translation of the base member along the control shaft causes the pivot member to pivot relative to the base member, and the first support to move relative to the second support.

19. The expandable implant of claim 18, wherein the recess is further defined by a second portion of the boundary wall, and wherein translational movement of the base member along the control shaft within the recess in a first direction is limited by the first portion of the boundary wall, and translational movement of the base member along the control shaft within the recess in a second direction is limited by the second portion of the boundary wall.

20. The expandable implant of claim 18, wherein the cap is configured to rotatably receive the control shaft to permit rotational movement of the control shaft and vertically fix the control shaft relative to the second support.
